(19) United States
(12) Patent Application Publication
(10) Pub. No.: US 2023/0337931 A1
(43) Pub. Date: Oct. 26, 2023

(54) SYSTEM AND METHOD FOR MAPPING REPOLARIZATION OF CARDIAC TISSUE

(71) Applicant: St. Jude Medical, Cardiology Division, Inc., St. Paul, MN (US)

(72) Inventor: Don Curtis Deno, Andover, MN (US)

(21) Appl. No.: 18/305,811

(22) Filed: Apr. 24, 2023

Related U.S. Application Data
(60) Provisional application No. 63/334,768, filed on Apr. 26, 2022.

(51) Int. Cl. A61B 5/0538; A61B 5/107

(52) U.S. Cl. CPC A61B 5/0538; A61B 5/1076

(57) ABSTRACT

A method of mapping cardiac tissue repolarization with an electroanatomical mapping system includes receiving electrophysiological data from a plurality of electrodes on a multi-electrode catheter. The electrodes define a plurality of cliques. For each clique, the electroanatomical mapping system can compute a vectorcardiogram including a depolarization loop and a repolarization loop, identify a depolarization time on the depolarization loop, define a repolarization interval, after the depolarization time, and identify a repolarization time, within the repolarization interval, on the repolarization loop. Over a plurality of beats and at different locations within the heart, this process creates a cardiac tissue repolarization map, which can be output graphically in various forms, including isochronal maps of repolarization time and/or activation recovery interval, representations of activation distance margin, and animated representations of propagating depolarization and repolarization wavefronts.

### CROSS-REFERENCE TO RELATED APPLICATIONS

This application claims the benefit of U.S. provisional application No. 63/334,768, filed 26 Apr. 2022, which is hereby incorporated by reference as though fully set forth herein.

### BACKGROUND

The present disclosure relates generally to electrophysiological mapping, as may be performed in cardiac diagnostic and therapeutic procedures. In particular, the present disclosure relates to systems, apparatuses, and methods for mapping and visualizing repolarization of electrophysiological tissue, including the use of data collected by a high density (“HD”) grid catheter or other multi-electrode device.

In healthy heart tissue, conduction velocities are relatively high and the time tissue spends in a depolarized state is relatively long. Thus, cardiac activation wavefronts extinguish themselves by running out of activatable myocardium and/or by colliding with recently-depolarized tissue.

On the other hand, it is understood that reentrant arrhythmias may occur when myocardial tissue repolarizes in time to depolarize, again, by a still-propagating wavefront (that is, the same cardiac activation wavefront activates the same myocardial tissue multiple times). This can result in a sustained, repetitive pattern of activation, which, in turn, can produce a tachycardia.

The ordinarily-skilled practitioner will appreciate that certain tachycardias cannot be tolerated for extended periods of time. It is therefore desirable to map cardiac tissue in a stable, well-tolerated rhythm (e.g., sinus rhythm or a paced rhythm) to determine locations potentially responsible for sustaining tachycardias, and thus suitable targets for ablation.

This type of substrate mapping can focus on identifying and ablating low-voltage or scar border zones, because it is understood that such regions may be involved in initiating or sustaining tachyarrhythmias. Another approach is to identify and ablate sites exhibiting late potentials—more precisely, local abnormal ventricular activity (LAVA) signals. It has also been proposed to identify and ablate areas of slow conduction.

There remain, however, various challenges associated with extant substrate analysis.

### BRIEF SUMMARY

The instant disclosure provides a method of mapping cardiac tissue repolarization, including: receiving, at an electroanatomical mapping system, electrophysiological data from a plurality of electrodes carried by a multi-electrode catheter, the plurality of electrodes defining a plurality of cliques; and for each clique of the plurality of cliques, the electroanatomical mapping system executing a process. The process executed by the electroanatomical mapping system includes the steps of: identifying a depolarization direction; identifying an omnipolar electrogram oriented along the depolarization direction; identifying a depolarization time in the omnipolar electrogram; defining a repolarization interval, occurring after the depolarization time, in the omnipolar electrogram; computing a vectorcardiogram repolarization loop over the repolarization interval; and identifying a repolarization time from the vectorcardiogram repolarization loop, thereby creating a cardiac tissue repolarization map.

In embodiments of the disclosure, the process executed by the electroanatomical mapping system for each clique of the plurality of cliques also includes defining a difference between the depolarization time and the repolarization time as an activation recovery interval for the clique. The cardiac tissue repolarization map can therefore include an activation recovery interval map.

In further embodiments of the disclosure, the process executed by the electroanatomical mapping system for each clique of the plurality of cliques also includes computing a conduction velocity for the clique and computing an activation distance margin for the clique as a product of the activation recovery interval for the clique and the conduction velocity for the clique. The cardiac tissue repolarization map can therefore include an activation distance margin map.

The step of defining the repolarization interval, occurring after the depolarization time, in the omnipolar electrogram, can include defining a start time for the repolarization interval, after the depolarization time, as a function of a cycle length. It can also include defining an end time for the repolarization interval, after the start time for the repolarization interval, as a function of the cycle length. Alternatively, the repolarization interval can be defined to have a preset duration, such as about 0.2 seconds.

The step of identifying the repolarization time from the vectorcardiogram repolarization loop can include defining a timing of an extreme location of the vectorcardiogram repolarization loop as the repolarization time. Alternatively, the step of identifying the repolarization time from the vectorcardiogram repolarization loop can include defining a timing at which the vectorcardiogram repolarization loop crosses a vectorcardiogram depolarization loop as the repolarization time.

It is contemplated that the multi-electrode catheter can be a high density grid catheter.

The method can also include outputting a graphical representation of the cardiac tissue repolarization map. The graphical representation of the cardiac tissue repolarization map can include an isochronal representation of the repolarization times for the plurality of cliques, an isochronal representation of activation recovery intervals for the plurality of cliques, a graphical representation of activation distance margins for the plurality of cliques, and/or an animated representation of both a cardiac activation wavefront, computed using the depolarization times for the plurality of cliques, and a cardiac repolarization wavefront, computed using the repolarization times for the plurality of cliques.

Also disclosed herein is an electroanatomical mapping system for generating a cardiac tissue repolarization map. The system includes a repolarization mapping and visualization processor configured to receive electrophysiological data from a plurality of electrodes carried by a multi-electrode catheter, the plurality of electrodes defining a plurality of cliques; and for each clique of the plurality of cliques, execute a process that includes: identifying a depolarization direction; identifying an omnipolar electrogram oriented along the depolarization direction; identifying a depolarization time in the omnipolar electrogram; defining a repolarization interval, occurring after the depolarization time, in the omnipolar electrogram; computing a vectorcardiogram repolarization loop over the repolarization interval; and identifying a repolarization time from the vectorcardiogram repolarization loop, thereby creating a cardiac tissue repolarization map.

The repolarization mapping and visualization processor can also be configured to output a graphical representation of the cardiac tissue repolarization map. In embodiments of the disclosure, the graphical representation of the cardiac tissue repolarization map can include an isochronal representation of the repolarization times for the plurality of cliques; an isochronal map of activation recovery intervals for the plurality of cliques; a graphical representation of activation distance margins for the plurality of cliques; and/or an animated representation of both a cardiac activation wavefront, computed using the depolarization times for the plurality of cliques, and a cardiac repolarization wavefront, computed using the repolarization times for the plurality of cliques.

The activation recovery interval for a clique can be defined as the difference between the depolarization time for the clique and the repolarization time for the clique.

The activation distance margin for a clique can be defined through the process executed by the repolarization mapping and visualization processor also including: computing a conduction velocity for the clique; and computing the activation distance margin for the clique as a product of the activation recovery interval for the clique and the conduction velocity for the clique.

The instant disclosure also provides a method of mapping cardiac tissue repolarization, including receiving, at an electroanatomical mapping system, electrophysiological data from a plurality of electrodes carried by a multi-electrode catheter, the plurality of electrodes defining a plurality of cliques; and for each clique of the plurality of cliques, the electroanatomical mapping system executing a process comprising: computing a vectorcardiogram including a depolarization loop and a repolarization loop; identifying a depolarization time on the depolarization loop; defining a repolarization interval, occurring after the depolarization time; and identifying a repolarization time, occurring within the repolarization interval, on the repolarization loop, thereby creating a cardiac tissue repolarization map.

In still other embodiments, the instant disclosure relates to an electroanatomical mapping system for generating a cardiac tissue repolarization map, including a repolarization mapping and visualization processor configured to receive electrophysiological data from a plurality of electrodes carried by a multi-electrode catheter, the plurality of electrodes defining a plurality of cliques; and for each clique of the plurality of cliques, execute a process including: computing a vectorcardiogram including a depolarization loop and a repolarization loop; identifying a depolarization time on the depolarization loop; defining a repolarization interval, occurring after the depolarization time; and identifying a repolarization time, occurring within the repolarization interval, on the repolarization loop, thereby creating a cardiac tissue repolarization map.

The foregoing and other aspects, features, details, utilities, and advantages of the present invention will be apparent from reading the following description and claims, and from reviewing the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram of an exemplary electroanatomical mapping system.

FIG. 2 depicts an exemplary catheter that can be used in connection with aspects of the instant disclosure.

FIGS. 3A and 3B provide alphanumeric labeling conventions for electrodes carried by a multi-electrode catheter and the bipoles associated therewith.

FIG. 4 is a flowchart of representative steps that can be carried out according to aspects of the instant disclosure.

FIG. 5A is an exemplary vectorcardiogram.

FIG. 5B is a magnified view of region B in FIG. 5A.

FIGS. 6A-6C illustrate, in two dimensions, depolarization and partial repolarization activity in healthy myocardium.

FIGS. 7A-7C illustrate, in two dimensions, depolarization and repolarization activity in diseased myocardium during a reentrant ventricular tachycardia.

FIGS. 8A-8F illustrate, in two dimensions, depolarization and repolarization activity highlighting a region that presents a risk of sustaining an arrhythmia.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

The instant disclosure provides systems, apparatuses, and methods for generating and visualizing electrophysiology maps, and in particular maps of cardiac tissue repolarization. For purposes of illustration, aspects of the disclosure will be described with reference to various cardiac repolarization maps, as created from intracardiac electrograms collected using a high density (HD) grid catheter, such as the Advisor™ HD grid mapping catheter from Abbott Laboratories (Abbott Park, Illinois), in conjunction with an electroanatomical mapping system, such as the EnSite Precision™ cardiac mapping system, also from Abbott Laboratories. Those of ordinary skill in the art will understand, however, how to apply the teachings herein to good advantage in other contexts and/or with respect to other devices.

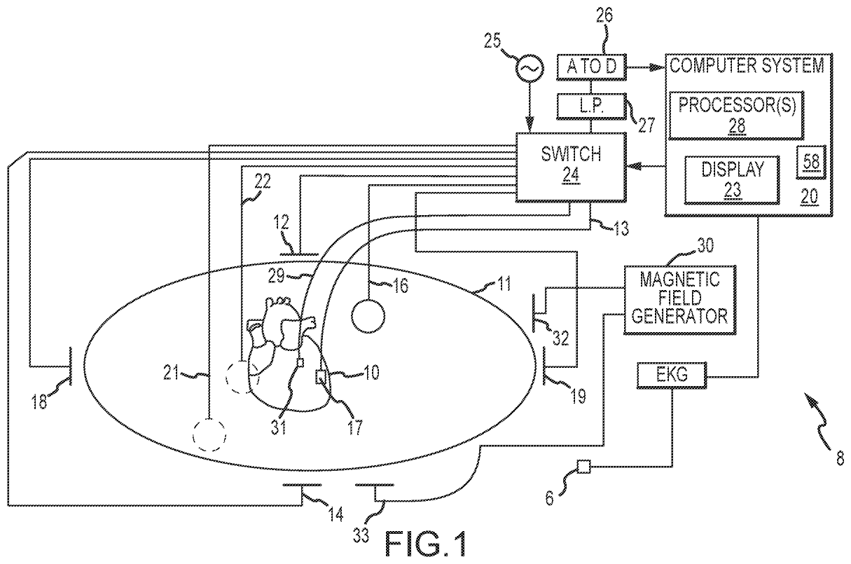

FIG. 1 shows a schematic diagram of an exemplary electroanatomical mapping system 8 for conducting cardiac electrophysiology studies by navigating a cardiac catheter and measuring electrical activity occurring in a heart 10 of a patient 11 and three-dimensionally mapping the electrical activity and/or information related to or representative of the electrical activity so measured. System 8 can be used, for example, to create an anatomical model of the patient's heart 10 using one or more electrodes. System 8 can also be used to measure electrophysiology data at a plurality of points along a cardiac surface and store the measured data in association with location information for each measurement point at which the electrophysiology data was measured, for example to create a diagnostic data map of the patient's heart 10.

As one of ordinary skill in the art will recognize, system 8 determines the location, and in some aspects the orientation, of objects, typically within a three-dimensional space, and expresses those locations as position information determined relative to at least one reference. This is referred to herein as “localization.”

For simplicity of illustration, the patient 11 is depicted schematically as an oval. In the embodiment shown in FIG. 1, three sets of surface electrodes (e.g., patch electrodes) 12, 14, 16, 18, 19, and 22 are shown applied to a surface of the patient 11, pairwise defining three generally orthogonal axes, referred to herein as an x-axis (12, 14), a y-axis (18, 19), and a z-axis (16, 22). In other embodiments the electrodes could be positioned in other arrangements, for example multiple electrodes on a particular body surface. As a further alternative, the electrodes do not need to be on the body surface, but could be positioned internally to the body.

In FIG. 1, the x-axis surface electrodes 12, 14 are applied to the patient along a first axis, such as on the lateral sides of the thorax region of the patient (e.g., applied to the patient's skin underneath each arm) and may be referred to as the Left and Right electrodes. The y-axis electrodes 16, 22 are applied to the patient along a second axis generally orthogonal to the x-axis, along the sternum and spine of the patient in the thorax region, and may be referred to as the Chest and Back electrodes. The z-axis electrodes 18, 19 are applied along a third axis generally orthogonal to both the x-axis and the y-axis, such as along the inner thigh and neck regions of the patient, and may be referred to as the Left Leg and Neck electrodes. The heart 10 lies between these pairs of surface electrodes 12/14, 16/22, and 18/19.

Each surface electrode can measure multiple signals. For example, in embodiments of the disclosure, each surface electrode can measure three resistance (impedance) signals and three reactance signals. These signals can, in turn, be grouped into three resistance/reactance signal pairs. One resistance/reactance signal pair can reflect driven values, while the other two resistance/reactance signal pairs can reflect non-drive values (e.g., measurements of the electric field generated by other driven pairs in a manner similar to that described below for electrodes 17).

An additional surface reference electrode (e.g., a “belly patch”) 21 provides a reference and/or ground electrode for the system 8. The belly patch electrode 21 may be an alternative to a fixed intra-cardiac electrode 31, described in further detail below. In alternative embodiments where system 8 is capable of magnetic field-based localization instead of or in addition to impedance-based localization, the surface electrode 21 can alternatively or additionally include a magnetic patient reference sensor—anterior (“PRS-A”) positioned on the patient's chest.

It should be appreciated that patient 11 may also have most or all of the conventional electrocardiogram (“ECG” or “EKG”) system leads in place. In certain embodiments, for example, a standard set of 12 ECG leads may be utilized for sensing electrocardiograms on the patient's heart 10. This ECG information is available to system 8 (e.g., it can be provided as input to computer system 20). Insofar as ECG leads are well understood, and for the sake of clarity in the figures, only a single lead 6 and its connection to computer 20 is illustrated in FIG. 1.

A representative catheter 13 having at least one electrode 17 is also shown. This representative catheter electrode 17 is referred to as the “roving electrode,” “moving electrode,” or “measurement electrode” throughout the specification. Typically, multiple electrodes 17 on catheter 13, or on multiple such catheters, will be used. In one embodiment, for example, the system 8 may comprise sixty-four electrodes on twelve catheters disposed within the heart and/or vasculature of the patient. In other embodiments, system 8 may utilize a single catheter that includes multiple (e.g., eight) splines, each of which in turn includes multiple (e.g., eight) electrodes.

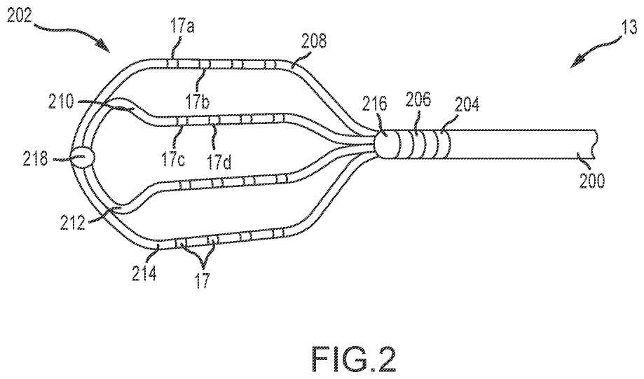

The foregoing embodiments are merely exemplary, however, and any number of electrodes and/or catheters may be used. For example, for purposes of this disclosure, a segment of an exemplary multi-electrode catheter, and in particular an HD grid catheter 13 such as the Abbott Laboratories Advisor™ HD Grid Mapping Catheter, Sensor Enabled™, is shown in FIG. 2.

HD grid catheter 13 includes a catheter body 200 coupled to a paddle 202. Catheter body 200 can further include first and second body electrodes 204, 206, respectively. Paddle 202 can include a first spline 208, a second spline 210, a third spline 212, and a fourth spline 214, which are coupled to catheter body 200 by a proximal coupler 216 and to each other by a distal coupler 218. In one embodiment, first spline 208 and fourth spline 214 can be one continuous segment and second spline 210 and third spline 212 can be another continuous segment. In other embodiments, the various splines 208, 210, 212, 214 can be separate segments coupled to each other (e.g., by proximal and distal couplers 216, 218, respectively). It should be understood that HD catheter 13 can include any number of splines; the four-spline arrangement shown in FIG. 2 is merely exemplary.

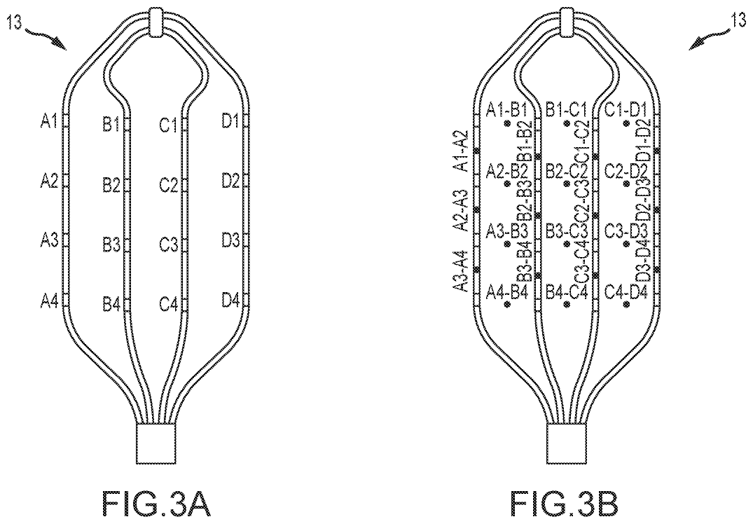

As described above, splines 208, 210, 212, 214 can include any number of electrodes 17; in FIG. 2, sixteen electrodes 17 are shown arranged in a four-by-four array. It should also be understood that electrodes 17 can be evenly and/or unevenly spaced, as measured both along and between splines 208, 210, 212, 214. For purposes of easy reference in this description, FIG. 3A provides alphanumeric labels for electrodes 17.

As those of ordinary skill in the art will recognize, any two neighboring electrodes 17 define a bipole. Thus, the 16 electrodes 17 on catheter 13 define a total of 42 bipoles −12 along splines (e.g., between electrodes 17a and 17b, or between electrodes 17c and 17d), 12 across splines (e.g., between electrodes 17a and 17c, or between electrodes 17b and 17d), and 18 diagonally between splines (e.g., between electrodes 17a and 17d, or between electrodes 17b and 17c).

For ease of reference in this description, FIG. 3B provides alphanumeric labels for the along- and across-spline bipoles. FIG. 3B omits alphanumeric labels for the diagonal bipoles, but this is only for the sake of clarity in the illustration. It is expressly contemplated that the teachings herein can also be applied with respect to the diagonal bipoles.

Any bipole can, in turn, be used to generate a bipolar electrogram according to techniques that will be familiar to those of ordinary skill in the art. Moreover, these bipolar electrograms can be combined to generate electrograms in any orientation of the plane of catheter 13.

For example, for the clique including electrodes C2, D2, and C3, the electrogram v_{θ}(t) in any orientation θ of the plane of catheter 13 can be computed according to the equation v_{θ}(t)=cos θ·vₓ(t)+sin θ·v_{y}(t), where vₓ(t) is the bipolar electrogram across splines (e.g., bipole C2-D2) and v_{y}(t) is the bipolar electrogram along the spline (e.g., bipole C2-C3). United States patent application publication no. 2018/0296111 (the '111 publication), which is hereby incorporated by reference as though fully set forth herein, discloses additional details of computing an E-field loop for a clique of electrodes on a HD grid catheter.

For purposes of description, electrograms v_{θ}(t) are referred to herein as “omnipolar electrograms” or “virtual bipolar electrograms.” These omnipolar electrograms can be thought of as the bipolar electrogram that would be seen by an “omnipole” or “virtual bipole” having its “omnipole orientation” or “virtual bipole orientation” at an angle θ relative to a catheter x-y coordinate system and in the plane of electrodes 17 of catheter 13.

In any event, catheter 13 can be used to simultaneously collect a plurality of electrophysiology data points for the various bipoles defined by electrodes 17 thereon, with each such electrophysiology data point including both localization information (e.g., position and orientation of a selected bipole) and an electrogram signal for the selected bipole. For purposes of illustration, methods according to the instant disclosure will be described with reference to individual electrophysiology data points collected by catheter 13. It should be understood, however, that the teachings herein can be applied, in serial and/or in parallel, to multiple electrophysiology data points collected by catheter 13 (e.g., over a plurality of cliques for a given position of catheter 13 within the heart, as well as for various positions of catheter 13 within the heart).

Catheter 13 (or multiple such catheters) are typically introduced into the heart and/or vasculature of the patient via one or more introducers and using familiar procedures. Indeed, various approaches to introduce catheter 13 into a patient's heart, such as transseptal approaches, will be familiar to those of ordinary skill in the art, and therefore need not be further described herein.

Since each electrode 17 lies within the patient, location data may be collected simultaneously for each electrode 17 by system 8. Similarly, each electrode 17 can be used to gather electrophysiological data from the cardiac surface (e.g., endocardial electrograms). The ordinarily skilled artisan will be familiar with various modalities for the acquisition and processing of electrophysiology data points (including, for example, both contact and non-contact electrophysiological mapping), such that further discussion thereof is not necessary to the understanding of the techniques disclosed herein. Likewise, various techniques familiar in the art can be used to generate graphical representations of cardiac geometry and/or cardiac electrical activity from the plurality of electrophysiology data points. Moreover, insofar as the ordinarily skilled artisan will appreciate how to create electrophysiology maps from electrophysiology data points, the aspects thereof will only be described herein to the extent necessary to understand the present disclosure.

Returning now to FIG. 1, in some embodiments, an optional fixed reference electrode 31 (e.g., attached to a wall of the heart 10) is shown on a second catheter 29. For calibration purposes, this electrode 31 may be stationary (e.g., attached to or near the wall of the heart) or disposed in a fixed spatial relationship with the roving electrodes (e.g., electrodes 17), and thus may be referred to as a “navigational reference” or “local reference.” The fixed reference electrode 31 may be used in addition or alternatively to the surface reference electrode 21 described above. In many instances, a coronary sinus electrode or other fixed electrode in the heart 10 can be used as a reference for measuring voltages and displacements; that is, as described below, fixed reference electrode 31 may define the origin of a coordinate system.

Each surface electrode is coupled to a multiplex switch 24, and the pairs of surface electrodes are selected by software running on a computer 20, which couples the surface electrodes to a signal generator 25. Alternately, switch 24 may be eliminated and multiple (e.g., three) instances of signal generator 25 may be provided, one for each measurement axis (that is, each surface electrode pairing).

The computer 20 may comprise, for example, a conventional general-purpose computer, a special-purpose computer, a distributed computer, or any other type of computer. The computer 20 may comprise one or more processors 28, such as a single central processing unit (“CPU”), or a plurality of processing units, commonly referred to as a parallel processing environment, which may execute instructions to practice the various aspects described herein.

Generally, three nominally orthogonal electric fields are generated by a series of driven and sensed electric dipoles (e.g., surface electrode pairs 12/14, 16/22, and 18/19) in order to realize catheter navigation in a biological conductor. Alternatively, these orthogonal fields can be decomposed and any pairs of surface electrodes can be driven as dipoles to provide effective electrode triangulation. Likewise, the electrodes 12, 14, 18, 19, 16, and 22 (or any number of electrodes) could be positioned in any other effective arrangement for driving a current to or sensing a current from an electrode in the heart. For example, multiple electrodes could be placed on the back, sides, and/or belly of patient 11. Additionally, such non-orthogonal methodologies add to the flexibility of the system. For any desired axis, the potentials measured across the roving electrodes resulting from a predetermined set of drive (source-sink) configurations may be combined algebraically to yield the same effective potential as would be obtained by simply driving a uniform current along the orthogonal axes.

Thus, any two of the surface electrodes 12, 14, 16, 18, 19, 22 may be selected as a dipole source and drain with respect to a ground reference, such as belly patch 21, while the unexcited electrodes measure voltage with respect to the ground reference. The roving electrodes 17 placed in the heart 10 are exposed to the field from navigational currents and are measured with respect to ground, such as belly patch 21. In practice the catheters within the heart 10 may contain more or fewer electrodes than the sixteen shown, and each electrode potential may be measured. As previously noted, at least one electrode may be fixed to the interior surface of the heart to form a fixed reference electrode 31, which is also measured with respect to ground, such as belly patch 21, and which may be defined as the origin of the coordinate system relative to which system 8 measures positions. Data sets from each of the surface electrodes, the internal electrodes, and the virtual electrodes may all be used to determine the location of the roving electrodes 17 within heart 10.

The measured voltages may be used by system 8 to determine the location in three-dimensional space of the electrodes inside the heart, such as roving electrodes 17 relative to a reference location, such as reference electrode 31. That is, the voltages measured at reference electrode 31 may be used to define the origin of a coordinate system, while the voltages measured at roving electrodes 17 may be used to express the location of roving electrodes 17 relative to the origin. In some embodiments, the coordinate system is a three-dimensional (x, y, z) Cartesian coordinate system, although other coordinate systems, such as polar, spherical, and cylindrical coordinate systems, are contemplated.

As should be clear from the foregoing discussion, the data used to determine the location of the electrode(s) within the heart is measured while the surface electrode pairs impress an electric field on the heart. The electrode data may also be used to create a respiration compensation value used to improve the raw location data for the electrode locations as described, for example, in U.S. Pat. No. 7,263,397, which is hereby incorporated herein by reference in its entirety. The electrode data may also be used to compensate for changes in the impedance of the body of the patient as described, for example, in U.S. Pat. No. 7,885,707, which is also incorporated herein by reference in its entirety.

Therefore, in one representative embodiment, system 8 first selects a set of surface electrodes and then drives them with current pulses. While the current pulses are being delivered, electrical activity, such as the voltages measured with at least one of the remaining surface electrodes and in vivo electrodes, is measured and stored. Compensation for artifacts, such as respiration and/or impedance shifting, may be performed as indicated above.

In aspects of the disclosure, system 8 can be a hybrid system that incorporates both impedance-based (e.g., as described above) and magnetic-based localization capabilities. Thus, for example, system 8 can also include a magnetic source 30, which is coupled to one or more magnetic field generators. In the interest of clarity, only two magnetic field generators 32 and 33 are depicted in FIG. 1, but it should be understood that additional magnetic field generators (e.g., a total of six magnetic field generators, defining three generally orthogonal axes analogous to those defined by patch electrodes 12, 14, 16, 18, 19, and 22) can be used without departing from the scope of the present teachings. Likewise, those of ordinary skill in the art will appreciate that, for purposes of localizing catheter 13 within the magnetic fields so generated, can include one or more magnetic localization sensors (e.g., coils).

In some embodiments, system 8 is the EnSite™ X, EnSite™ Velocity™, or EnSite Precision™ electrophysiological mapping and visualization system of Abbott Laboratories. Other localization systems, however, may be used in connection with the present teachings, including for example the RHYTHMIA HDX™ mapping system of Boston Scientific Corporation (Marlborough, Massachusetts), the CARTO navigation and location system of Biosense Webster, Inc. (Irvine, California), the AURORA® system of Northern Digital Inc. (Waterloo, Ontario), Stereotaxis, Inc.'s NIOBE® Magnetic Navigation System (St. Louis, Missouri), as well as MediGuide™ Technology from Abbott Laboratories.

The localization and mapping systems described in the following patents (all of which are hereby incorporated by reference in their entireties) can also be used with the present invention: U.S. Pat. Nos. 6,990,370; 6,978,168; 6,947,785; 6,939,309; 6,728,562; 6,640,119; 5,983,126; and 5,697,377.

Aspects of the disclosure relate to generating electrophysiology maps, and in particular to mapping the repolarization of tissue. Graphical representations of such electrophysiology maps can also be output, for example on display 23. System 8 can therefore include a repolarization mapping and visualization module 58.

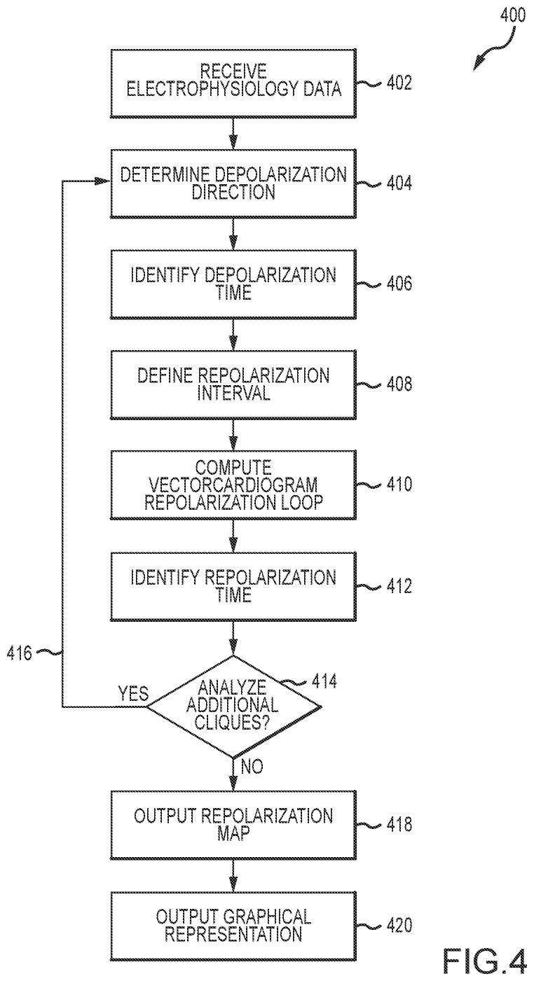

Exemplary methods according to aspects of the instant disclosure, which use cardiac repolarization mapping as illustrative, will be explained with reference to the flowchart of representative steps presented as FIG. 4. In some embodiments, for example, flowchart 400 may represent several exemplary steps that can be carried out by electroanatomical mapping system 8 of FIG. 1 (e.g., by processor 28 and/or repolarization mapping and visualization module 58). It should be understood that the representative steps described below can be hardware-implemented, software-implemented, or implemented in a combination of hardware and software.

In block 402, system 8 receives electrophysiological data measured by electrodes 17 on catheter 13. As discussed above, electrodes 17 define a plurality of electrode cliques, with each clique including three (or, in some embodiments of the disclosure, four or more) electrodes.

Blocks 404 through 412 represent a series of steps carried out by system 8 with respect to a plurality of such cliques and, in certain aspects of the disclosure, with respect to all such cliques on catheter 13.

In block 404, system 8 identifies a depolarization direction for the respective clique. According to particular embodiments of the disclosure, the depolarization direction for the respective clique corresponds to the omnipolar orientation at which the omnipolar electrogram for the clique reaches maximum amplitude (that is, the angle θ that maximizes the amplitude of v_{Θ}(t)). Identification of the orientation of the maximum amplitude omnipolar electrogram is described, for example, in United States patent application publication no. 2020/0077908, which is hereby incorporated by reference as though fully set forth herein.

In other embodiments of the disclosure, the depolarization direction for the respective clique corresponds to the cardiac activation direction for the clique. Those of ordinary skill in the art will be familiar with various approaches to identifying the cardiac activation direction for a clique of electrodes. By way of example only, however, one suitable approach is described in United States patent application publication no. 2021/0361215, which is hereby incorporated by reference as though fully set forth herein.

In certain instances, the two foregoing approaches to identifying the depolarization direction for the clique may yield the same, or substantially the same (e.g., within about 4 degrees of each other) depolarization direction.

As one of ordinary skill in the art will appreciate from the foregoing disclosure, the depolarization direction is associated with a particular omnipolar electrogram. Indeed, as described above, certain embodiments of the disclosure identify the depolarization direction because of the characteristics of this omnipolar electrogram (e.g., it is the maximum amplitude omnipolar electrogram for the clique). Further analysis, as described below, is conducted on this electrogram oriented along the depolarization direction.

In block 406, for example, system 8 identifies a depolarization time (denoted herein as T_{D}) in the omnipolar electrogram oriented along the depolarization direction. The ordinarily-skilled artisan will recognize numerous suitable ways to identify a depolarization time in an electrogram signal, including identifying the time where the absolute value of the first derivative of the electrogram signal is maximized. Other suitable approaches to identifying depolarization time in an electrogram signal are described in international patent application publication no. WO/2020/242940, which is hereby incorporated by reference as though fully set forth herein.

In block 408, system 8 defines a repolarization interval. Logically, both the start time and the end time of the repolarization interval occur temporally after the depolarization time identified in block 406.

The purpose of defining the repolarization interval is to provide a more limited window within which system 8 will analyze the electrogram signal to identify the repolarization time (denoted herein as T_{R}). That is, rather than analyzing the entire electrogram signal to identify the repolarization time, system 8 analyzes only the repolarization interval to identify the repolarization time. Advantageously, therefore, the repolarization interval need not be defined with extreme precision. Instead, it need only be defined wide enough to encompass the repolarization time.

As such, in embodiments of the disclosure, system 8 defines the start time of the repolarization interval as a function of cycle length. For instance, a time step 6 can be defined using an adaptation of Fridericia's formula for correcting a cycle length-dependent QT interval:

$$δ = 0.35 \cdot \sqrt[3]{CL} ,$$

where CL is the cycle length of the preceding beat.

Alternatively, δ can be defined using an adaptation of the Framingham formula for correcting the QT interval: δ=0.196+0.154·CL, where CL is the cycle length of the preceding beat.

In still further aspects, δ can be defined using an adaptation of another accepted approach to correcting the QT interval, such as Hodges' formula or Bazett's formula.

Once the time step is computed, the start time of the repolarization interval can be defined as T_{D}+δ.

In certain embodiments of the disclosure, the repolarization interval has a preset width, such as about 0.2 seconds. It is also contemplated that system 8 may permit a practitioner to adjust the width of the repolarization window.

In other embodiments of the disclosure, the end time of the repolarization window is defined as a function of cycle length. For example, where the start time of the repolarization window is computed using an adaptation of Fridericia's formula as described above, the end time of the repolarization window can also be computed utilizing an adaptation of Fridericia's formula:

$${T}_{D} + 0.46 \cdot \sqrt[3]{CL} .$$

Likewise, where the start time of the repolarization window is computed using an adaptation of the Framingham formula as described above, the end time of the repolarization window can also be computed using an adaptation of the Framingham formula: T_{D}+0.306+0.154·CL.

From the foregoing description, those of ordinary skill in the art will appreciate how to adapt other formulae to compute the end time of the repolarization window.

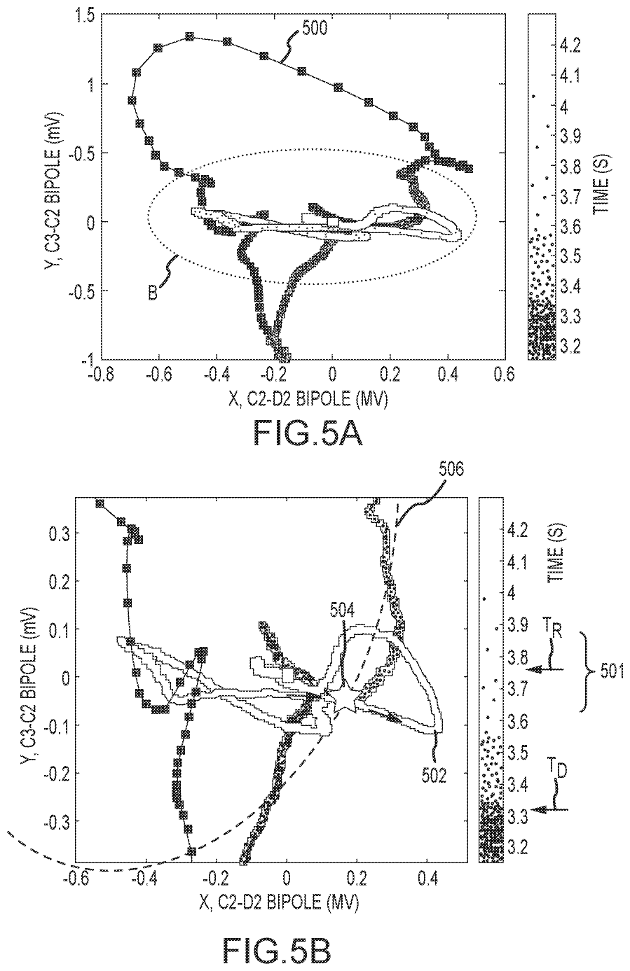

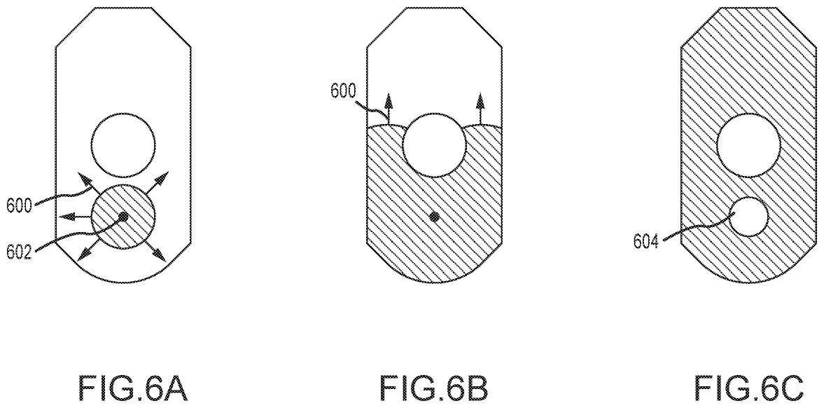

In block 410, system 8 computes a vectorcardiogram repolarization loop over the cardiac cycle (which, of course, includes the repolarization interval). Those of ordinary skill in the art will be familiar with the computation of vectorcardiograms, such that a detailed description of the same is not required for an understanding of the instant disclosure. For purposes of illustration, however, FIG. 5A depicts an exemplary vectorcardiogram 500 of the two-dimensional electrogram for clique C2-D2-C3, while FIG. 5B is a magnified version of region B in FIG. 5A. The depolarization time T_{D} and repolarization interval 501 are also annotated in FIG. 5B.

It should be understood that vectorcardiogram 500 illustrates the vectorcardiogram for an entire beat, not just the repolarization interval. For visualization purposes, however, the colorscale used in FIGS. 5A and 5B presents the depolarization loop portion of the vectorcardiogram in blue to violet and the repolarization loop portion of the vectorcardiogram in green to red.

System 8 analyzes the vectorcardiogram repolarization loop over the repolarization interval to identify the repolarization time in block 412. The instant disclosure contemplates various approaches to identifying the repolarization time.

According to one embodiment of the disclosure, system 8 defines an extreme location of the vectorcardiogram repolarization loop as the repolarization time. An extreme location may be designated as the time in the repolarization interval that is the maximum of the second derivative of the loop trajectory. This can be expressed mathematically, for example, as

$${T}_{R} = {max}_{t} \{ \vert \frac{{d}^{2} {v}_{x} ( t )}{{dt}^{2}} \vert + \vert \frac{{d}^{2} {v}_{y} ( t )}{{dt}^{2}} \vert \} or {T}_{R} = {max}_{t} \{ {( \frac{{d}^{2} {v}_{x} ( t )}{{dt}^{2}} )}^{2} + {( \frac{{d}^{2} {v}_{y} ( t )}{{dt}^{2}} )}^{2} \} .$$

Referring to FIG. 5B, for instance, system 8 could define extreme point 502 as the repolarization time.

According to another embodiment of the disclosure, system 8 defines a point at which the repolarization loop portion of the vectorcardiogram crosses the depolarization loop portion of the vectorcardiogram as the repolarization time. For instance, referring to FIG. 5B, system 8 could define crossing point 504 as the repolarization time. The use of subintervals, for example as disclosed in international patent application publication no. WO/2020/242940, may be particularly desirable when defining the region of the depolarization loop that should be analyzed to identify the crossing point of the repolarization loop.

According to aspects of the disclosure, system 8 can compensate for minor variations at the beginning and end of a depolarization by fitting a quadratic to the first third and last third of a time interval centered on the local depolarization. This improves robustness and consistency in the definition of the repolarization time. An exemplary quadratic fit is represented by dashed line 506 in FIG. 5B.

Decision block 414, including loopback 416 (through the “YES” exit from decision block 414), allows system 8 to analyze additional cliques. Once all cliques are analyzed (through the “NO” exit from decision block 414), system 8 can output a data set including repolarization times for a plurality of cliques in block 418. For simplicity, this data set, as collected over one or more beats, is referred to herein as a “cardiac tissue repolarization map.”

A graphical representation of the cardiac tissue repolarization map can be output in block 420. The present disclosure contemplates a number of possible graphical representations.

For instance, in some embodiments of the disclosure, the cardiac tissue repolarization map can be output as a colorscale, greyscale, pattern scale, or isochronal representation of repolarization times T_{R} over a geometric model of the heart in a manner analogous to known graphical representations of local activation times (that is, graphical representations of cardiac depolarization activity).

Activation recovery intervals (“ARIs”), which can be computed as the difference between T_{D} and T_{R} for a given clique, can be represented similarly.

Repolarization time and/or ARI, standing alone, can provide a first order indicator of possible arrythmias. A more refined indicator of possible arrythmias considers both repolarization time or ARI and conduction velocity.

Thus, aspects of the disclosure relate to computing activation distance margins (“ADMs”) for respective cliques. More particularly, the ADM for a clique can be computed as the product of the ARI for the clique and the conduction velocity for the clique. Because those of ordinary skill in the art will be familiar with computing conduction velocities for cliques of electrodes, a detailed description of the same need not be provided herein. By way of example only, however, U.S. Pat. No. 9,808,171 and United States patent application publication no. 2021/0361215, each of which is hereby incorporated by reference as though fully set forth herein, describe suitable approaches to computing conduction velocity. ADMs can be represented graphically in a manner analogous to other electrophysiology characteristics (e.g., as a colorscale, greyscale, or pattern scale representation of values over a geometric model of the heart).

Still further, system 8 can output a graphical representation of a cardiac repolarization wavefront, which can be computed from the repolarization times. Techniques analogous to those used to output graphical representation of cardiac activation (that is, depolarization) wavefronts can be applied to generate the graphical representation of the cardiac repolarization wavefront.

In yet further embodiments of the disclosure, system 8 can output a graphical representation that combines both an animated representation of a cardiac activation wavefront and an animated representation of a cardiac repolarization wavefront as the respective wavefronts propagate over the cardiac surface. Once again, various techniques are known to output animated representations of cardiac activation wavefronts, and analogous techniques can be applied to output animated representations of cardiac repolarization wavefronts.

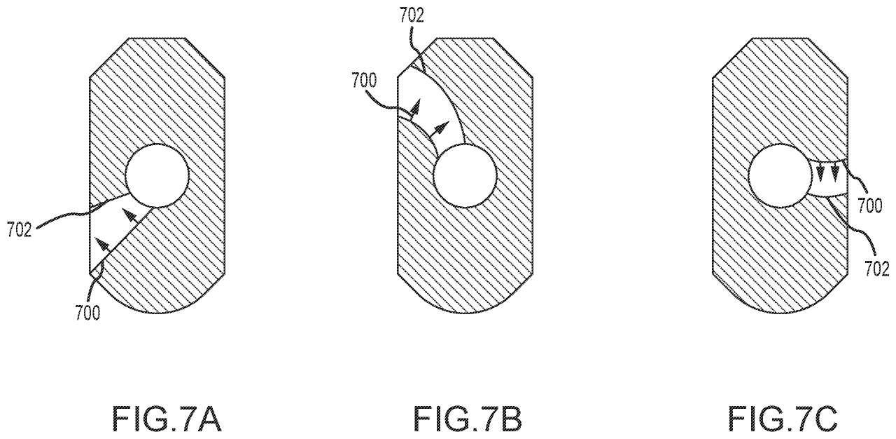

Such combined animations may be advantageous to a practitioner in visualizing arrhythmias and/or target tissues for ablation. For instance, FIGS. 6A-8F illustrate (in two-dimensions, for ease of understanding) representations of depolarization and repolarization activity in the left ventricle. In each of FIGS. 6A-8F, the aortic valve is at the top, the left ventricular apex is at the bottom, and the mitral valve orifice is in the center. Refractory tissue (that is, tissue that has depolarized, but not yet repolarized) is shown in cross-hatching. Non-refractory tissue is shown without cross-hatching.

FIGS. 6A-6C depict depolarization and repolarization activity in healthy cardiac tissue. In FIG. 6A, which represents the left ventricle at a time t₁, the depolarization wavefront 600 has begun to spread from a site of initial activation 602. Depolarization wavefront 600 continues to spread as shown in FIG. 6B, which represents the left ventricle at a later time t₂. At a still later time t₃, as shown in FIG. 6C, depolarization of the left ventricle is complete, and tissue behind repolarization wavefront 604 has begun to repolarize.

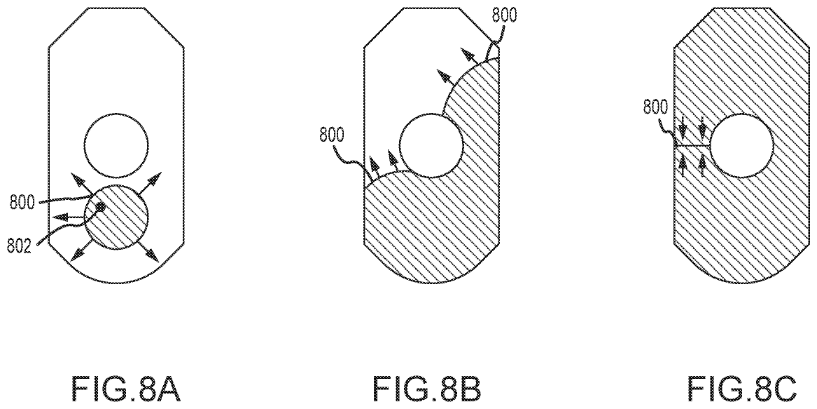

FIGS. 7A-7C depict depolarization and repolarization activity in diseased myocardium during a reentrant ventricular tachycardia at successive, but not necessarily consecutive, times t₁, t₂, and t₃, respectively. As shown in FIGS. 7A-7C, there is always a depolarization wavefront 700 spreading forwards and a retreating repolarization wavefront 702 just ahead of depolarization wavefront 700, creating a circular pathway that sustains a reentrant ventricular tachycardia.

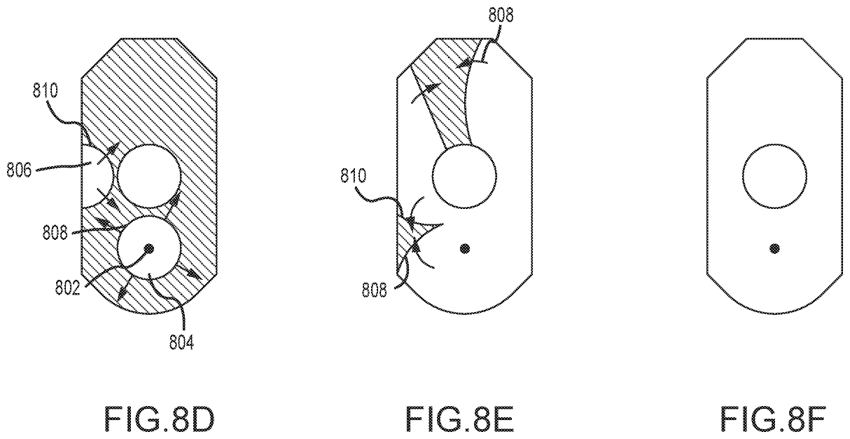

FIGS. 8A-8G also depict depolarization and repolarization activity in myocardium that presents a risk of reentrant arrhythmia at successive, but not necessarily immediately sequential, times t₁ through t₇, respectively. In FIG. 8A, the depolarization wavefront 800 has begun to spread from the site of initial activation 802, much as shown in FIG. 6A. Unlike in FIGS. 6A-6C, however, in FIGS. 8A-8G, the depolarization wavefront moves much more quickly on one side of the mitral valve than the other, as shown in FIG. 8B, ultimately colliding with itself as shown in FIG. 8C.

In FIG. 8D, two regions 804, 806 have begun to repolarize, with one repolarization wavefront 808 spreading from site of initial activation 802 after a relatively longer ARI (e.g., from time t₁ to time t₄) and a separate repolarization wavefront 810 spreading in a critical isthmus with after a relatively shorter ARI (e.g., from time t₃ to time t₄). Further repolarization from time t₄ to time t₅ is reflected in FIG. 8E. Finally, in FIG. 8F, all tissue has been repolarized.

Viewing FIGS. 8A-8F as sequential frames in an animation will emphasize the relatively short period of time that region 806 spends depolarized. This shorter ARI in region 806 indicates a risk of arrythmia and an incipient unidirectional block.

Although several embodiments have been described above with a certain degree of particularity, those skilled in the art could make numerous alterations to the disclosed embodiments without departing from the spirit or scope of this invention.

For example, the teachings herein can be applied in real time (e.g., during an electrophysiology study) or during post-processing (e.g., to electrophysiology data points collected during an electrophysiology study performed at an earlier time).

As another example, it is contemplated that the depolarization time T_{D} and the repolarization time T_{R} can be defined directly from the vectorcardiogram (e.g., without first identifying the depolarization direction and the associated omnipolar electrogram). Approaches to identifying the repolarization time T_{R} in a vectorcardiogram are described above. One suitable approach to identifying the depolarization time T_{D} in a vectorcardiogram is to look for the time t where the distance between regularly sampled points is greatest. This is the two dimensional analogue of finding the time an ordinary one dimensional signal has maximum slope. Mathematically, this can be expressed as

$${T}_{D} = {max}_{t} \{ \vert \frac{{dv}_{x} ( t )}{dt} \vert + \vert \frac{{dv}_{y} ( t )}{dt} \vert \} or {max}_{t} \{ {( \frac{{dv}_{x} ( t )}{dt} )}^{2} + {( \frac{{dv}_{y} ( t )}{dt} )}^{2} \} .$$

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A method of mapping cardiac tissue repolarization, comprising:
receiving, at an electroanatomical mapping system, electrophysiological data from a plurality of electrodes carried by a multi-electrode catheter, the plurality of electrodes defining a plurality of cliques; and
for each clique of the plurality of cliques, the electroanatomical mapping system executing a process comprising:
identifying a depolarization direction;
identifying an omnipolar electrogram oriented along the depolarization direction;
identifying a depolarization time in the omnipolar electrogram;
defining a repolarization interval, occurring after the depolarization time, in the omnipolar electrogram;
computing a vectorcardiogram repolarization loop over the repolarization interval; and
identifying a repolarization time from the vectorcardiogram repolarization loop, thereby creating a cardiac tissue repolarization map.

2. The method according to claim 1, wherein the process executed by the electroanatomical mapping system for each clique of the plurality of cliques further comprises defining a difference between the depolarization time and the repolarization time as an activation recovery interval for the clique.

3. The method according to claim 2, wherein the cardiac tissue repolarization map comprises an activation recovery interval map.

4. The method according to claim 2, wherein:
the process executed by the electroanatomical mapping system for each clique of the plurality of cliques further comprises:
computing a conduction velocity for the clique; and
computing an activation distance margin for the clique as a product of the activation recovery interval for the clique and the conduction velocity for the clique, and
the cardiac tissue repolarization map comprises an activation distance margin map.

5. The method according to claim 1, wherein defining the repolarization interval, occurring after the depolarization time, in the omnipolar electrogram, comprises defining a start time for the repolarization interval, after the depolarization time, as a function of a cycle length.

6. The method according to claim 5, wherein defining the repolarization interval, occurring after the depolarization time, in the omnipolar electrogram comprises defining an end time for the repolarization interval, after the start time for the repolarization interval, as a function of the cycle length.

7. The method according to claim 1, wherein defining the repolarization interval, occurring after the depolarization time, in the omnipolar electrogram, comprises defining the repolarization interval to have a preset duration.

8. The method according to claim 7, wherein the preset duration of the repolarization interval is 0.2 seconds.

9. The method according to claim 1, wherein identifying the repolarization time from the vectorcardiogram repolarization loop comprises defining a timing of an extreme location of the vectorcardiogram repolarization loop as the repolarization time.

10. The method according to claim 1, wherein identifying the repolarization time from the vectorcardiogram repolarization loop comprises defining a timing at which the vectorcardiogram repolarization loop crosses a vectorcardiogram depolarization loop as the repolarization time.

11. The method according to claim 1, wherein the multi-electrode catheter comprises a high density grid catheter.

12. The method according to claim 1, further comprising outputting a graphical representation of the cardiac tissue repolarization map.

13. The method according to claim 12, wherein the graphical representation of the cardiac tissue repolarization map comprises an isochronal representation of the repolarization times for the plurality of cliques.

14. The method according to claim 12, wherein the graphical representation of the cardiac tissue repolarization map comprises an isochronal representation of activation recovery intervals for the plurality of cliques.

15. The method according to claim 12, wherein the graphical representation of the cardiac tissue repolarization map comprises a graphical representation of activation distance margins for the plurality of cliques.

16. The method according to claim 12, wherein the graphical representation of the cardiac tissue repolarization map comprises an animated representation of both a cardiac activation wavefront, computed using the depolarization times for the plurality of cliques, and a cardiac repolarization wavefront, computed using the repolarization times for the plurality of cliques.

17. An electroanatomical mapping system for generating a cardiac tissue repolarization map, comprising:
a repolarization mapping and visualization processor configured to:
receive electrophysiological data from a plurality of electrodes carried by a multi-electrode catheter, the plurality of electrodes defining a plurality of cliques; and
for each clique of the plurality of cliques, execute a process comprising:
identifying a depolarization direction;
identifying an omnipolar electrogram oriented along the depolarization direction;
identifying a depolarization time in the omnipolar electrogram;
defining a repolarization interval, occurring after the depolarization time, in the omnipolar electrogram;
computing a vectorcardiogram repolarization loop over the repolarization interval; and
identifying a repolarization time from the vectorcardiogram repolarization loop,
thereby creating a cardiac tissue repolarization map.

18. The electroanatomical mapping system according to claim 17, wherein the repolarization mapping and visualization processor is further configured to output a graphical representation of the cardiac tissue repolarization map.

19. The electroanatomical mapping system according to claim 18, wherein the graphical representation of the cardiac tissue repolarization map comprises an isochronal representation of the repolarization times for the plurality of cliques.

20. The electroanatomical mapping system according to claim 18, wherein the graphical representation of the cardiac tissue repolarization map comprises an animated representation of both a cardiac activation wavefront, computed using the depolarization times for the plurality of cliques, and a cardiac repolarization wavefront, computed using the repolarization times for the plurality of cliques.

21. The electroanatomical mapping system according to claim 17, wherein the process executed by the repolarization mapping and visualization processor for each clique of the plurality of cliques further comprises defining a difference between the depolarization time and the repolarization time as an activation recovery interval for the clique.

22. The electroanatomical mapping system according to claim according to claim 21, wherein:
the process executed by the repolarization mapping and visualization processor for each clique of the plurality of cliques further comprises:
computing a conduction velocity for the clique; and
computing an activation distance margin for the clique as a product of the activation recovery interval for the clique and the conduction velocity for the clique, and
the cardiac tissue repolarization map comprises an activation distance margin map.

23. A method of mapping cardiac tissue repolarization, comprising:
receiving, at an electroanatomical mapping system, electrophysiological data from a plurality of electrodes carried by a multi-electrode catheter, the plurality of electrodes defining a plurality of cliques; and
for each clique of the plurality of cliques, the electroanatomical mapping system executing a process comprising:
computing a vectorcardiogram including a depolarization loop and a repolarization loop;
identifying a depolarization time on the depolarization loop;
defining a repolarization interval, occurring after the depolarization time; and
identifying a repolarization time, occurring within the repolarization interval, on the repolarization loop,
thereby creating a cardiac tissue repolarization map.

24. An electroanatomical mapping system for generating a cardiac tissue repolarization map, comprising:
a repolarization mapping and visualization processor configured to:
receive electrophysiological data from a plurality of electrodes carried by a multi-electrode catheter, the plurality of electrodes defining a plurality of cliques; and
for each clique of the plurality of cliques, execute a process comprising:
computing a vectorcardiogram including a depolarization loop and a repolarization loop;
identifying a depolarization time on the depolarization loop;
defining a repolarization interval, occurring after the depolarization time; and
identifying a repolarization time, occurring within the repolarization interval, on the repolarization loop,
thereby creating a cardiac tissue repolarization map.
